(19) United States
(12) Patent Application Publication
(10) Pub. No.: US 2006/0129096 A1
(43) Pub. Date: Jun. 15, 2006

(54) Fluid-handling device

(76) Inventor: Alastair Douglas Wright, Central Scotland (GB)

Correspondence Address: BAKER & DANIELS LLP;111 E. WAYNE STREET, SUITE 800, FORT WAYNE, IN, 46802, US

(21) Appl. No.: 10/524,163

(22) PCT Filed: Sep. 25, 2003
(86) PCT No.: PCT/GB03/04174

§ 371 Date: Feb. 7, 2005

(30) Foreign Application Priority Data
Sep. 25, 2002 (GB) 0222167.9

(51) Int. Cl. A61M 5/00; A61M 5/32

(52) U.S. Cl. 604/110; 604/192

(57) ABSTRACT

A pre-filled hypodermic syringe adapted for use with a retractable-type needle unit (18). The syringe has a barrel (10) provided with a piston member (14). The piston member (14) includes a plastics portion (30) mounting a dislodgeable blocking portion (36) of chemically inert material in such a way that the plastics portion (30) is not in contact with the pre-filled contents of the syringe.

This invention relates to a fluid-handling device, in particular, a hypodermic syringe, and is particularly concerned with devices which are pre-filled with a drug or other component to be dispensed so that the device may be stored and then used subsequently.

When fluids such as drugs are to be stored for any length of time, it is important that there is no risk of contamination by the materials that they are in contact with during storage. Thus, for pre-filled applications, the device typically comprises a barrel made of glass having a dispensing outlet closed with a bung of silicone rubber and a piston member of silicone rubber located in the barrel for discharging the pre-filled contents of the barrel after the bung has been removed and the dispensing outlet of the barrel has been fitted with an injection needle.

The present invention seeks to provide an improved fluid-handling device which is particularly for pre-filled applications and is adapted to accommodate retraction of the needle into the barrel after use.

In accordance with a first aspect of the present invention, there is provided a pre-filled hypodermic syringe adapted for use with a retractable-type needle unit, the syringe having a barrel provided with a piston member which includes a plastics portion mounting a dislodgeable blocking portion of chemically inert material in such a way that the plastics portion is not in contact with the pre-filled contents of the syringe.

In accordance with a second aspect of the present invention, there is provided a fluid-handling device comprising a barrel having a closable dispensing outlet at one end, and a piston member insertable into the barrel to form, forwardly thereof a chamber within the barrel which can be pre-filled with the component to be dispensed, movement of the piston member towards the dispensing outlet being effective to force the chamber contents, in use, through the dispensing outlet when open, the piston member comprising a rim portion, a blocking portion mounted by the rim portion and severable from the latter, and a seal for making sealing engagement with the internal wall of the barrel, the arrangement being such that the rim portion is not exposed to the contents of said chamber and the forward side of blocking portion is presented within the chamber for co-operation with a retractable-type needle unit when fitted to the forward end of the barrel.

The term “retractable-type needle unit” as used herein means a needle unit adapted for fitting to a syringe barrel and comprising an injection needle which projects from a housing of the unit for the purpose of administering injections to a patient but is thereafter retractable, usually automatically, into the barrel so that the needle is then inaccessible, at least to the exent necessary to avoid needle stick injuries. Re-use of the needle may also be prevented by rendering the needle inaccessible within the barrel.

Severance (partial or complete) of the blocking portion from the rim portion during needle retraction allows the needle to pass through the piston member and fully enter the barrel where it is no longer accessible.

The rim portion may be of a plastics material. While, it is considered undesirable for plastics materials to be in contact with drug components under storage conditions because of the risk of contamination, the rim portion is so arranged that it is not exposed to the contents of the chamber thereby allowing the use of a material which does not possess the high degree of chemical inertness associated with glass and silicone rubber.

By employing a rim portion of plastics material, the piston member may comprise a blocking portion provided with an overmoulded rim portion, e.g. by an insert moulding technique in which the rim portion is moulded around an insert formed by the blocking portion, which may be of glass.

The blocking portion and the rim portion may be engaged with one another in such a way that the blocking portion is severed or dislodged from the rim portion upon application of an appropriate axial force, e.g. that exerted by a retractable needle driven by a biasing element (e.g. a spring) following administration of an injection.

In one embodiment of the invention, the forwardly facing surface of the rim portion is covered by a material, such as silicone rubber, which is acceptable for long term contact with the drug or other component to be stored.

The material covering the rim portion may be formed by an integral extension of the seal which is typically of silicone rubber. The seal may be formed as a separate moulding or it may be moulded around the rim portion.

For storage purposes, the dispensing outlet of the barrel may be fitted with a bung or the like.

Typically the seal is made of an elastomeric material, such as silicone rubber, which is acceptable for use in pre-filled applications.

For dispensing purposes, the dispensing outlet of the barrel may be fitted with a retractable-type needle unit.

The rim portion may be in the form of a sleeve receiving the blocking portion and the arrangement may be such that the silicone rubber covering overlies the forward end of the sleeve to prevent contact with the chamber contents.

The rim portion may include an annular section having a perimetral groove for location of the seal.

Forward movement of the piston member within the barrel may be effected by a rod which may be separate from the piston member so that the rod need only be inserted into the barrel when required to effect dispensing.

The rim portion may be adapted to locate the forward end of the rod.

The arrangement may be such that when the dispensing stroke of the rod has been completed, its rear end is rendered substantially inaccessible or captive with the barrel so that the rod cannot then be pulled back.

To this end, the rear end of the rod may be provided with a head against which thumb pressure may be applied during the dispensing stroke and, upon completion of the dispensing stroke, the head may engage in a retainer provided at the rear end of the barrel, e.g. so that the head is engaged as an interference fit or wedged into the retainer. If desired, co-operating formations such as ratchet teeth may be provided on the head and/or the retainer to prevent withdrawal of the head from the retainer and hence rearward movement of the rod.

The rod may be hollow so that the needle can enter into its interior following triggering of needle retraction.

The needle unit may be adapted to make snap fit engagement with the dispensing outlet of the barrel.

The dispensing outlet may be a necked down part of the barrel.

The needle unit may include a coupling member for engagement with the dispensing outlet of the barrel.

The needle retraction mechanism may be as disclosed in our prior International Patent Application No. PCT/GB02/01865 or our co-pending UK Patent Application entitled “Needle Unit” of even date herewith, the entire contents of both applications being incorporated herein by this disclosure.

The invention will now be described by way of example only with reference to the accompanying drawings, in which:

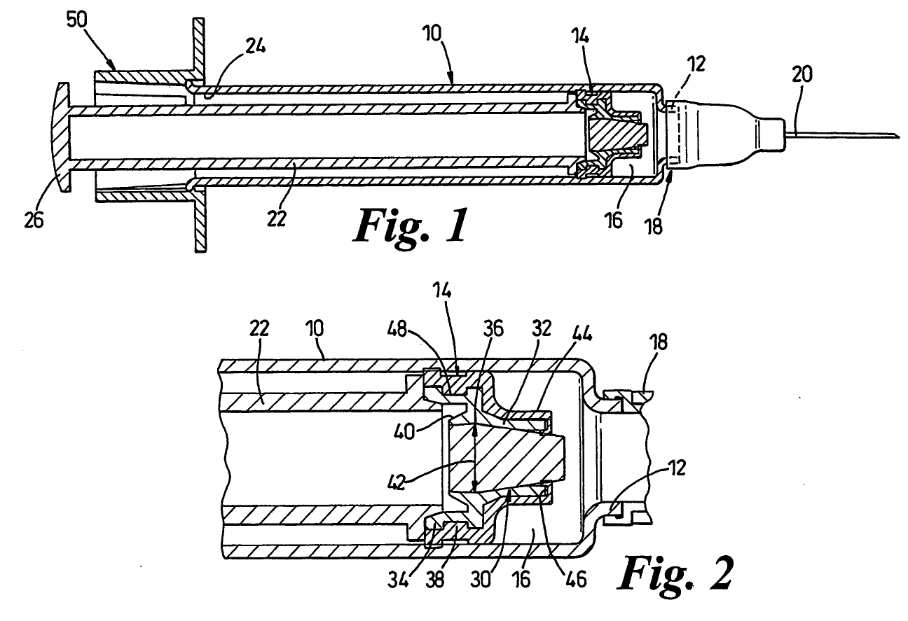

FIG. 1 is a longitudinal sectional view of a hypodermic syringe suitable for pre-filled applications, the piston member of the barrel being shown partially displaced along the barrel during the dispensing stroke of the syringe;

FIG. 2 is an enlarged view in section of the syringe in FIG. 1. Referring now to the drawings, the syringe comprises a glass barrel 10 which is open at both ends, the forward end being formed with a necked portion 12 forming a dispensing outlet of the barrel. The barrel 10 acccommodates a piston member 14 which is movable axially. In its pre-filled condition, the outlet 12 is closed, e.g. with a silicone rubber bung (not shown) and the piston member 14 forms together with the portion of the barrel forward thereof a chamber 16 which is filled with the drug or other component to be stored, e.g. relatively long term, awaiting subsequent administration to a patient. In the drawings, the piston member 14 is shown partly advanced during a dispensing stroke. It will be understood that, in the pre-filled, storage condition, the piston member 14 will be located further from the outlet 12 and the chamber defined will be filled with the drug or other component.

When the contents of the chamber 16 are to be dispensed, the bung is removed and the forward end of the barrel 10 is fitted with an injection needle unit 18 so that the bore of the needle 20 is in communication with the chamber 16. A rod 22 is inserted into the barrel via its rear end 24 and engaged with the rear side of the piston member 14. During the injection procedure pressure is applied, e.g. using the thumb, to the head 26 of the rod 22 to displace the piston member 14 towards the outlet 12 so as to force the contents of the chamber 16 through the needle.

The needle unit 18 is of the retractable-type in which, following delivery of the drug or other component, retraction of the needle is automatically triggered so that the needle is driven rearwardly into the barrel 10. Retraction-type needle units are well known in the art. Typically the barrel in the illustrated embodiment may be used in conjunction with a retraction-type needle unit as disclosed in International Patent Application No. PCT/GB02/01865 or our co-pending UK Patent Application entitled “Needle Unit” of even date herewith. The needle unit 18 may have a snap engagement with the outlet 12 or it may be fitted in some other fashion, e.g. by a screw-threaded connection or a connection which prevents subsequent removal of the needle unit from the barrel—e.g. using a connection as disclosed in UK Patent No. 2353078.

To allow the use of a retractable-type needle unit, the piston member 14 is adapted to enable the needle to be driven fully into the interior of the barrel 10, and more particularly into the rod 22 which is hollow for this purpose. The piston member 14 comprises an annular rim portion 30 comprising a central tubular sleeve 32 and an annular section 34, a blocking portion 36 which is captive with the rim portion 30 in such a way as to be severable therefrom, and an annular seal 38 which sealingly engages with the internal wall of the barrel 10.

The blocking portion 36 is in the form of a solid plug of glass, a material which is acceptable for long term contact with the drug or other component. Likewise the seal 38 is composed of a material which is acceptable for long term contact with the drug or other component, e.g. a suitable elastomeric material such as silicone rubber. The rim portion 30 however is a plastics material such as polypropylene, which is somewhat less chemically inert with respect to typical drug components than glass or silicone rubber.

By using a plastics material for this component, it is possible to render the plug 36 captive but severable in a simple and effective manner, namely by moulding the rim portion 30 around the plug 36 using an insert moulding technique such that the plug 36 forms part of the moulding surface. As will be seen from FIG. 2, the rim portion 30 is moulded about the plug 36 and the components are shaped so that the plug is captive but can be released by application of a suitable rearwardly directed axial force suffient to push the plug past the collar portion 40 of the rim portion 36. It will be appreciated that there will be some degree of resilient deformation of the plastics material involved as the enlarged mid-section 42 passes through the collar portion 40. In practice, the design is such that the force acting on the needle during retraction is sufficient to dislodge the plug 36 and allow the needle to pass through the rim portion 30 into the interior of the rod 22.

The use of a plastics material in this environment however is generally considered unacceptable because of the risk of the contents of the chamber 16 being contaminated, e.g. by leaching of ingredients from the plastics material. This is overcome in the illustrated embodiment by covering the otherwise exposed rim portion with an inert material so that all surfaces at the front side of the piston member which are exposed to the interior of the chamber 16 are of an inert material. This is achieved in a particularly effective manner by producing the seal 38 with an extension 44 which overlies the rim portion 30 and forms an additional seal at the interface between the plug 36 and the forward end 46 of the rim portion. For example, the silicone rubber seal 38 and its extension 44 may be moulded around the combined assembly of the plug and the rim portion.

The seal 38 is located on the rim portion by a groove 48 in the annular section 34. The annular section 34 also serves to locate the forward end of the hollow rod 22 which seats within the section 34. The rear end of the barrel 10 is provided with a collar 50 designed to co-operate with the head 26 of the rod in such a way that, once the dispensing stroke of the rod 22 has been effected, the head 26 engages within the collar 50 and can no longer be gripped by the fingers. The arrangement may be such that the head 26 is rendered captive with the collar 50 by any suitable means, e.g. interference fit or ratchet formations, thereby retaining the rod in its forward position in which it safely encloses the needle.

Whilst endeavouring in the foregoing specification to draw attention to those features of the invention believed to be of particular importance, it should be understood that the Applicant claims protection in respect of any patentable feature or combination of features disclosed herein and/or shown in the drawings whether or not particular emphasis has been placed on such feature or features.

1. A pre-filled hypodermic syringe adapted for use with a retractable-type needle unit, the syringe having a barrel provided with a piston member which includes a plastics portion mounting a dislodgeable blocking portion of chemically inert material in such a way that the plastics portion is not in contact with the pre-filled contents of the syringe.

2. A fluid-handling device comprising a barrel having a closable dispensing outlet at one end, and a piston member insertable into the barrel to form forwardly thereof a chamber within the barrel which can be pre-filled with the component to be dispenses, movement of the piston member towards the dispensing outlet being effective to force the chamber contents, in use, through the dispensing outlet when open, the piston member comprising a rim portion, a blocking portion mounted by the rim portion and severable from the latter, and a seal for making sealing engagement wit the internal wall of the barrel, the arrangement being such that the rim portion is not exposed to the contents of said chamber and the forward side of blocking portion is presented within the chamber for co-operation with a retractable-type needle unit when fitted to the forward end of the barrel.

3. A fluid-handling device according to claim 2, wherein at least partial severance of the blocking portion from the rim portion during needle retraction allows the needle to pass through the piston member and fully enter the barrel.

4. A fluid-handling device according to claim 2, wherein the rim portion is of a plastics material.

5. A fluid-handling device according to claim 4, wherein the piston member comprises a blocking portion provided with an overmolded rim portion.

6. A fluid-handling device according to claim 5, wherein the overmolded rim portion is formed by an insert molding technique in which the rim portion is molded around an insert formed by the blocking portion.

7. A fluid-handling device according to claim 2, wherein the blocking portion and the rim portion are engaged with one another in such a way that the blocking portion is severed or dislodged from the rim portion upon application of an appropriate axial force.

8. A fluid-handling device according to claim 7, wherein the axial force is exerted by the retractable needle driven by a biasing element following administration of an injection.

9. A fluid-handling device according to claim 2, wherein the forwardly facing surface of the rim portion is covered by a material that is acceptable for long term contact with the component to be stored.

10. A fluid-handling device according to claim 9, wherein the material covering the rim portion may be formed by an integral extension of the seal.

11. A fluid-handling device according to claim 10, wherein the rim portion may be in the form of a sleeve receiving the blocking portion and the arrangement is such that the covering material overlies the forward end of the sleeve to prevent contact with the chamber contents.

12. A fluid-handling device according to claim 2, wherein the rim portion includes an annular section having a perimetral groove for location of the seal.

13. A fluid-handling device according to claim 2, wherein forward movement of the piston member within the barrel is effected by a rod that is separate from the piston member.

14. A fluid-handling device according to claim 13, wherein the rim portion is adapted to locate the forward end of the rod.

15. A fluid-handling device according to claim 13, wherein the arrangement is such that when the dispensing stroke of the rod has been completed, its rear end is rendered substantially inaccessible or captive with the barrel.

16. A fluid-handling device according to claim 15, wherein the rear end of the rod is provided with a head against which thumb pressure may be applied during the dispensing stroke and, upon completion of the dispensing stroke, the head engages in a retainer provided at the rear end of the barrel.

17. A fluid-handling device according to claim 16, wherein co-operating formations are provided on the head and/or the retainer to prevent withdrawal of the head from the retainer.

18. A fluid-handling device according to claim 13, wherein the rod is hollow so that the needle can enter into its interior following triggering of needle retraction.

19. A fluid-handling device according to claim 2, wherein the needle unit is adapted to make snap fit engagement with the dispensing outlet of the barrel.

20. A fluid-handling device according to claim 2, wherein the dispensing outlet may be a necked down part of the barrel.

21. A fluid-handling device according to claim 2, wherein the needle unit includes a coupling member for engagement with the dispensing outlet of the barrel.
